(19) United States
(12) Patent Application Publication
(10) Pub. No.: US 2023/0172450 A1
(43) Pub. Date: Jun. 8, 2023

(54) SYSTEM AND METHOD FOR REMOTE MOTOR AND SENSORY FUNCTION TESTING

(71) Applicant: Christiana Care Health System, Inc., Wilmington, DE (US)

(72) Inventors: John DiGiovanni, Wilmington, DE (US); Jason Mastriana, Newark, DE (US)

(21) Appl. No.: 17/964,314

(22) Filed: Oct. 12, 2022

Related U.S. Application Data
(60) Provisional application No. 63/254,888, filed on Oct. 12, 2021.

(51) Int. Cl. A61B 5/00; G16H 40/67; A61B 5/11; G09B 19/00

(52) U.S. Cl. CPC A61B 5/0022; G16H 40/67; A61B 5/1124; A61B 5/4005; A61B 5/748; A61B 5/7455; G09B 19/003

(57) ABSTRACT

A system for remote motor and sensory function testing may involve use of patent and clinician computing devices, a patient testing device, and a clinician control device. A Remote Testing System (RTS) may coordinate displays of information among the devices in support of a test. A clinician or the RTS may cause display of instructions for guiding a patient in using the patient testing device. A clinician may receive feedback from the patient's interaction during the test session, or during a later session. The clinician control device may be used to control functioning of the patient testing device, in support of the test. The clinician control device may provide haptic or other feedback during the test session or during a later session. The system may track patient performance across multiple sessions and initiate follow-up test sessions as a result of data gathered in one or more prior test sessions.

### CROSS-REFERENCE TO RELATED APPLICATION

This application claims the benefit of priority of U.S. Provisional Patent Application No. 63/254,888, filed Oct. 12, 2021, the entire disclosure of which is hereby incorporated herein by reference.

### FIELD OF THE INVENTION

The present invention relates generally to computerized systems, and more specifically, to a computerized system and method permitting a clinician to remotely monitor and test a patient's motor and sensory functions.

### DISCUSSION OF RELATED ART

It is often difficult for patients with motor and/or sensory function problems to meet in person, e.g., at a hospital or other care facility, with a physician, nurse or other clinician for the purpose of testing of the patient's motor and sensory functions, which may be necessary for diagnosis and/or management of chronic conditions or disease states.

Some solutions may exist that use mobile device monitoring for some form of limited clinical assessment, but they are generally “once and done” types of tools that do not provide adequate tools for long-term condition/disease management over time.

What is needed is a way for a clinician to conduct a motor function and sensory examination when the patient is not physically present with the clinician that provides adequate tools for long-term condition/disease management over multiple sessions in a prolonged period of time.

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 is a schematic block diagram illustrating an exemplary and non-limiting embodiment of a Patient Computing Device in accordance with the present invention;

FIG. 3 is a schematic block diagram illustrating an exemplary and non-limiting embodiment of a Clinician Computing Device in accordance with the present invention;

FIG. 4 is a schematic block diagram illustrating an exemplary and non-limiting embodiment of a Patient Testing Device in accordance with the present invention;

FIG. 5 is a schematic block diagram illustrating an exemplary and non-limiting embodiment of a Clinician Control Device in accordance with the present invention;

FIG. 6 is a schematic block diagram illustrating an exemplary and non-limiting embodiment of a computerized Remote Testing System in accordance with the present invention; and

FIG. 7 is a flow diagram illustrating operation of the system in accordance with an exemplary and non-limited embodiment of the present invention.

### SUMMARY

The present invention provides a system and method allowing for remote testing of a patient's motor and/or sensory functions when the patient is not physically present with a clinician. Further, the system and method provide tools for long-term condition/disease management by actions taken during multiple sessions over a prolonged period of time.

A system for remote motor and sensory function testing comprises a memory operatively comprising a non-transitory data processor-readable medium, a data processor operatively connected to the memory, and user interface management instructions embodied in data processor-executable code stored in the memory. The user interface management instructions are executable by the data processor to provide an interface management engine configured to: cause display of instructions for performance of at least one of a motor function test and a sensory test at a remotely located computing device, the instructions comprising instructions for a test involving interacting with a patient testing device by physical contact with the patient testing device, the patient testing device comprising at least one sensor for gathering sensor data as a result of the physical contact; receive sensor data corresponding to the physical contact with the patient testing device; process the sensor data to generate feedback data, the feedback data indicating the patient's interaction with the patient testing device; and transmit the feedback data via a communications network.

The system may include instructions to store the feedback data in the memory, and the instructions to transmit the feedback data via the communications network may involves retrieving the feedback data from the memory, and wherein the said instructions cause the transmitting in response to a request for transmission of the feedback data at a time after completion of the at least one of the motor function test and the sensory test. Displays of instructions at a patient computing device for using a patient testing device and at a clinician computing device for using a clinician control device may be coordinated in concert in support of performance of the at least one of the motor function test and the sensory test. The instructions may cause display first instruction data at a first time for a first test and display of second instruction data at a second time for a second test, and the causing of display of second instruction data at the second time for the second test may be a result of sending of the second instruction data at the second time as determined automatedly according to data gathered during the first test.

### DETAILED DESCRIPTION

The present invention provides a computerized system that permits a clinician to conduct or review a motor function and/or sensory examination when the patient is not physically present with the clinician. The system may provide for repeated testing of a single patient over time, and may incorporates machine learning or other techniques to analyze results over time and passively transmit/push a personalized testing and/or care plan specific to the user, based on past results over a plurality of testing sessions of that same patient, to provide for long-term condition/disease management for that patient.

Accordingly, the present invention may be used by neurologists or other healthcare providers within and outside of health systems, by people with motor function and/or sensory impairments.

More particularly, the present invention provides a computerized system including patient testing device hardware for receiving inputs from a patient/user, and a graphical user interface that is controlled in concert with the patient testing device to allow a clinician (physician, nurse, etc.) to remotely conduct a motor function and sensory examination by having the patient (user) in a remote location position the patient's hands (or other body parts) in contact with a device that contains one or more joysticks, touchpoints, etc. relevant to conducting of the desired motor function and sensory examination tests. The graphical user interface may be included in the patient testing device, or may be provided in a distinct separate device (such as a conventional smartphone, tablet, laptop or personal computer device) that is controlled in concert with the patient testing device hardware.

For example, one or more joysticks of the patient testing device hardware may allow the user to push and pull their hands in different directions, which may send signals to the centrally-located computerized system and ultimately resulting signals or information to the clinician's device to deliver feedback as to how well the user is performing one or more specified functions.

By way of further example, the patient testing device's touchpoints may similarly allow the clinician to deliver hot or cold signals (to provided warmed or cooled surfaces at the touchpoints of the patient testing device) to the user for sensory testing. The system, via the graphical user interface, may then ask the patient if he/she can sense the hot or cold sensation. These touchpoints can also be positioned on different body parts, such as legs or arms.

In certain embodiments, the patient testing device hardware may run embedded software and be tethered via Bluetooth to a tablet computer or phone running custom software that controls the data communication between the user and clinician.

Similarly, the present invention provides a computerized system including optional clinician control device hardware for receiving inputs from a clinician, and/or a graphical user interface that is controlled in concert with the clinical control device and/or the patient testing device to allow the clinician to remotely conduct a motor function and sensory examination by having the patient (user) in the remote location position the patient's hands (or other body parts) in contact with the patient testing device to conduct the desired motor function and sensory examination tests. The graphical user interface may be included in the clinician control device, or may be provided in a distinct separate device (such as a conventional smartphone, tablet, laptop or personal computer device) that is controlled in concert with the clinician control and/or patient testing device hardware.

Accordingly, there may be a centrally-located computerized system in data communication providing graphical user interfaces to computerized devices physically located with the clinician and/or with the person/patient, and in data communication with patient testing device hardware physically located with the patient and/or clinician control device hardware physically located with the clinician.

In certain embodiments, the data forwarded to the physician computing device allows for the physician computing device to provide real-time feedback to the clinician, e.g., by displaying a representation of an intensity of by the patient's hand, as if the user were squeezing the clinician's hand, in a manner corresponding to the patient's squeezing input at the patient testing device.

In certain other embodiments, the system may provide haptic feedback to the clinician via clinician control device, to provide an opportunity for the clinician to haptically feel a recreation of the patient's input, e.g., equivalent pressure or tension. For example, data forwarded to the clinician control device may allow the clinician control device to provide real-time feedback to the clinician, e.g., by squeezing the clinician's hand, as if the user were squeezing the clinician's hand, in a manner corresponding to the patient's squeezing input at the patient testing device.

Additionally, the patient/user can use the patient testing device to perform motor and sensory function tests without the clinician's active involvement in a single joint session (in other words, patient and clinician sessions may be asynchronous), and data may be stored by the system for future use and analysis by the clinician. Accordingly, the clinician can select a specific date/time a user performed a test, and then receive/review associated data at another time, including haptic feedback from the clinician control device, according to how the data was recorded by the patient testing device during the user's actual time of taking the test.

Accordingly, remote motor function and sensory examination may be conducted using software and peripherals, in combination with a virtual (video, chat, call) communication channel.

In some embodiments, the testing device can be configured to be used as a stand-alone product for the patient/user to monitor the patient's own motor and sensory functions and track progress to report/discuss with a clinician at a later date.

The clinician control device and/or the Remote Testing System may be configured to transmit/push a personalized testing and/or care plan specific to the user, based on past results over a plurality of testing sessions, after analyzing data from a plurality of testing sessions.

The Remote Testing System may analyze test results, e.g., using artificial intelligence and/or machine learning techniques, and the results and/or result trends may trigger follow-up actions through artificial intelligence and notify the patient/user when to engage with a clinician and/or provide instructions for a next best step, etc. More particularly, as patients use the system and more data is collected from test performance over one or more sessions (such as how well the patient is performing, the duration of the test, where there is difficulty, etc.) suitable algorithms learn, not only from the patient's data but from data of other users of the system, and incorporate the clinical profile of the patient to perform an analysis for triggering follow-up actions. The outcome of this analysis can be used to make informed decisions on what test a specific patient should do next, when the patient might need to reach out to the patient's physician, and/or give the patient and/or the clinician guidance on next steps.

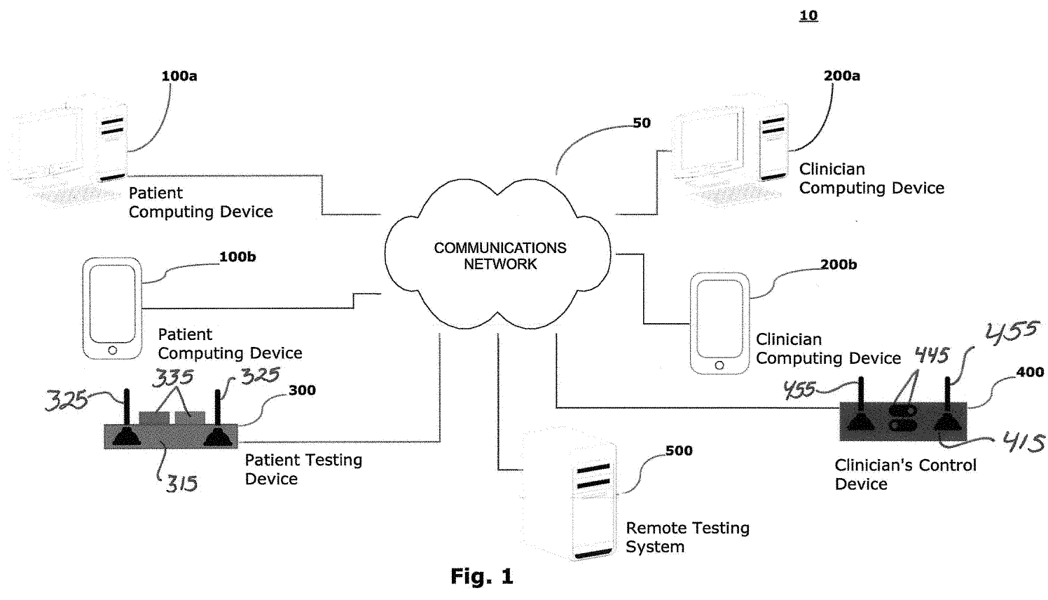

According to illustrative embodiment(s) of the present invention, various views are illustrated in FIGS. 1-5 and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

### System Environment

An exemplary embodiment of the present invention is discussed below for illustrative purposes. FIG. 1 is a system diagram showing an exemplary network computing environment 10 in which the present invention may be employed. As shown in FIG. 1, the exemplary network environment 10 includes certain conventional computing hardware and software for communicating via a communications network 50, such as the Internet, etc., using a Patient Computing Devices 100a, 100b and Clinician Computing Devices 200a, 200b, each of which may be, for example, one or more personal computers/PCs, laptop computers, tablet computers, smartphones, or other computing system hardware, including computerized/networked communication hardware/software/functionality, such as computer-based servers, kiosks and the like, or other so-called “connected” communication devices having communication capabilities for communicating data via the network and for delivering messages, in any form, to the user, such as smart watches, activity trackers, headphones, ear buds, televisions, or any other computerized and/or internet-of-things type device. By way of example, the term “messages” is used in a broad non-limiting fashion to include messages deliverable a user via such a communication device, such as an e-mail message, a text/SMS or similar “i-message”, as well as notifications such as a telephone ring, beep/tone or other audible alert, calendar reminders, alert messages and other displayable textual or graphic notifications designed to notify and/or communicate information to a user of the communication device.

In accordance with a certain aspect of the present invention, one or more of the Patient Computing Devices and/or Clinician Computing Devices 100a, 100b, 200a, 200b is a smartphone, tablet computer, smart watch or other computing device configured to store and execute an “app” or other purpose-specific application software in accordance with the present invention, although this is not required in all embodiments.

In accordance with another aspect of the present invention, the exemplary network environment 10 includes a Patient Testing Device 300 usable by a patient and capable of delivering stimuli to the user and/or receiving input from the user, that is relevant to a clinician's testing/assessment of the patient's motor and sensory functions, which may be necessary for diagnosis and/or management of chronic conditions or disease states. The Patient Testing Device 300 is further capable of capturing associated data operation of the Patient Testing Device 300 (e.g., the results of testing the patient's motor and sensory functions) and electronically communicating such data or related data to another device, e.g., via the communications network 50. By way of example, the Patient Testing Device 300 may include a discrete housing 315 such that the device is configured as a discrete, stand-alone device. The housing 315 may support one or more joysticks or levers 325 that are user-manipulable in fashions relevant to testing the of patient's motor functions. Further, for example, the Patient Testing Device 300 may include one or more touchpads 330 that may be selectively heated above or cooled below an ambient temperature in fashions relevant to testing of the patient's sensory functions.

By way of example, a touchpad may be caused to be heated by causing a flow of electricity through an associated resistive heating element adjacent the touchpad 330 that warms as a result of the flow of electricity. By way of further example, a touchpad may be caused to be cooled by causing a flow of cooled/refrigerated fluid to be circulated through passages adjacent the touchpad 330, or by releasing a liquid in a chamber adjacent the touchpad 330 that evaporates or sublimes upon release to result in cooling of the touchpad by absorbing heat from the touchpad by a process using the latent heat of vaporization of the liquid. Alternatively, for example, a Peltier semiconductor device may be used to provide a heating or cooling effect by suitable application of electrical current, as will be appreciated by those skilled in the art.

The Patient Testing Device 300 may include any suitable sensors, devices, structures, control circuitry and/or other mechanisms for delivering the controlled stimuli and/or receiving the desired inputs from the patient, and for communicating associated data as desired. By way of example, the Patient testing Device 300 may include tactile or haptic sensors, squeeze points, or audio output ports to provide a way of connecting noise canceling headphones for output used in hearing testing instead of and/or in addition to one or more joysticks and touchpads capable of delivering stimuli to the user, and/or receiving input from the user, that is relevant to a clinician's testing/assessment of the patient's motor and sensory functions.

In accordance with another aspect of the present invention, the exemplary network environment 10 includes a Clinician Control Device 400 usable by a clinician and capable of receiving inputs from a clinician to cause delivering of stimuli to the user via the Patient Testing Device 300, and/or delivering outputs/feedback to the clinician as a result of the patient's inputs provided at the Patient Testing Device 300, that are relevant to a clinician's testing/assessment of the patient's motor and sensory functions.

The Clinician Control Device 400 is further capable of capturing data associated with operation of the Clinician Control Device 400 (e.g., the results of the clinician's inputs to the Clinician Control Device) and of processing received data to provide feedback/stimuli and/or control operation of the Clinician Control Device 400 (e.g., to provide haptic feedback to the clinician via devices of the Clinician Control Device 400), and electronically communicating such data or related data to another device, e.g., via the communications network 50.

By way of example, the Clinician Control Device 400 may include a discrete housing 415 such that the device is configured as a discrete, stand-alone device.

The housing 415 may support one or more switches 445 that may be user-manipulable in fashions relevant to testing of the patient's motor and/or sensory functions at the Patient Testing Device 300. By way of example, one or more switches 445 may be provided to selectively cause a touchpad 330 to be heated or cooled at the Patient Testing Device 300.

Further, the housing 415 may support one or more joysticks or levers 455 that may be user-manipulable in fashions relevant to testing of the patient's motor and/or sensory functions and/or may be driven to provide haptic feedback to a user at the Clinician Control Device 400 based on patient input at the Patient Testing Device 300. By way of example, movement and/or pressure exerted by a Patient on the joysticks 325 of the Patient Testing Device 300 may be effectively replicated by driving/actuation of the joysticks/levers, a clamp, or other device at the Clinician Control Device, so that the clinician at the Clinician Control Device 400 can effectively feel/experience the actions of the patient at the Patient Testing Device 300. Accordingly, in addition to real-time sessions between the patient and the clinician, the system may provide for an asynchronous mode of interaction by which the patient interaction is recorded in a cloud computing infrastructure that can be used for later playback on the clinician device. Accordingly, instead of the clinician simply reviewing a textual test result report electronically, the clinician can interact with the Clinician Control Device to experience the patient's input and the associated test results as if they were happening in real-time.

The Clinician Control Device 400 may include any suitable sensors, devices, structures, control circuitry and/or other mechanisms for delivering the desired feedback/outputs and/or receiving the desired inputs from the clinician, and for communicating associated data as desired. By way of example, the Clinician Control Device 400 may include buttons or potentiometers to deliver signals to be used for various sensory tests including feeling feedback, hearing testing, etc. instead of and/or in addition to one or more joysticks/levels and switches capable of delivering feedback/output to the user, and/or receiving input from the user, that is relevant to a clinician's testing/assessment of the patient's motor and sensory functions.

In certain embodiments, each of the Patient Testing Device 300 and/or the Clinician Control Device 400 may include a graphical user interface or other display that are controlled in concert with a graphical user interface of other display of the Patient Testing Device 300 and/or the Clinician Control Device 400 and/or one or more of the Patient Computing Devices 100a, 100b and/or the Clinician Computing Devices 200a, 200b, to facilitate the clinician and/or patient in performing a remotely conducted motor function and sensory examination.

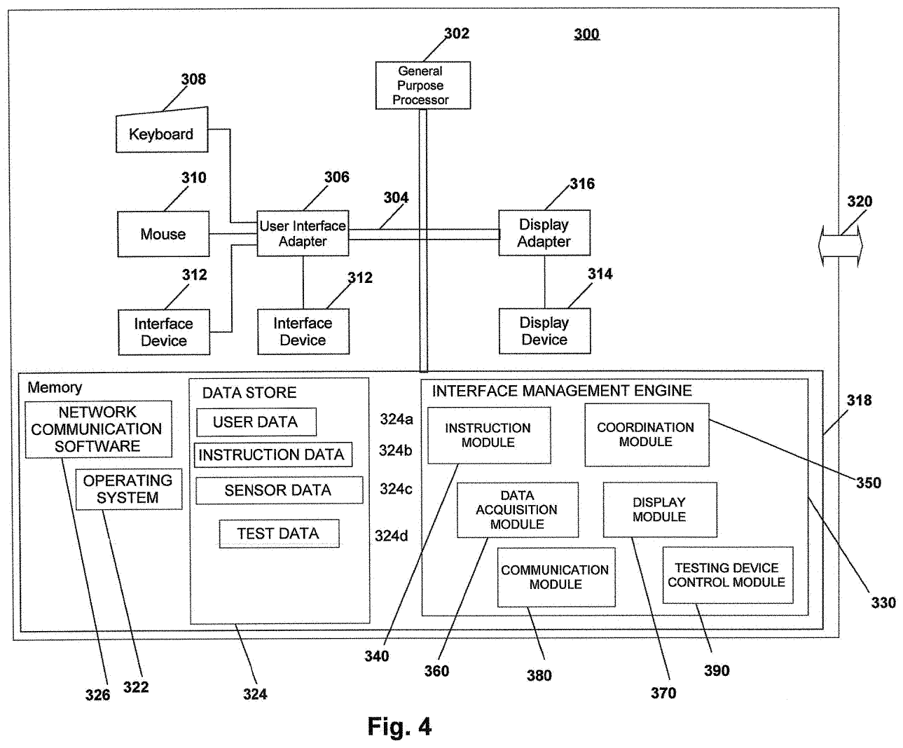

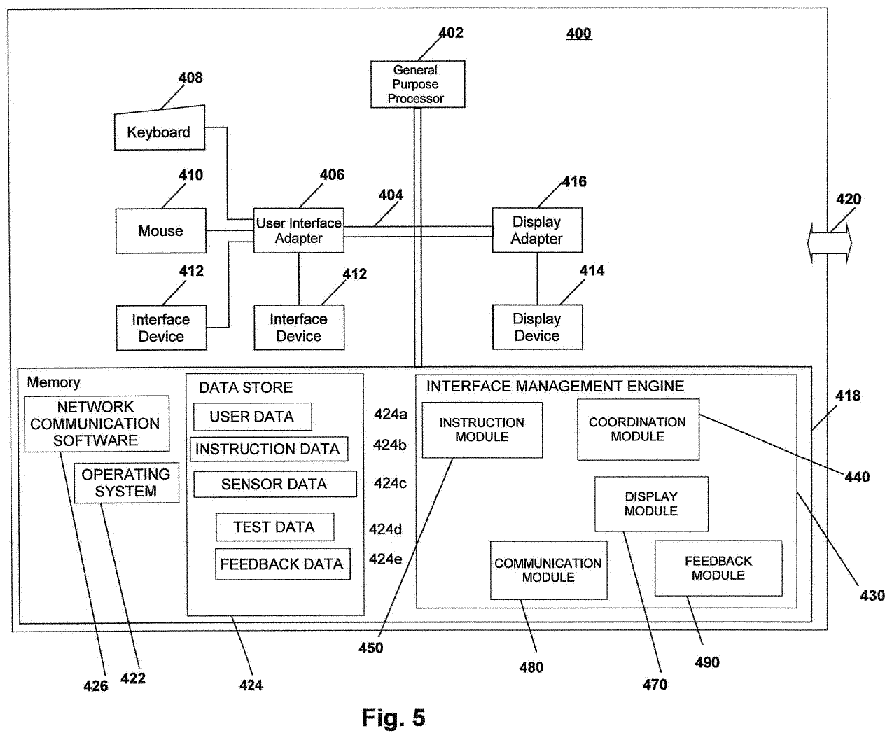

In some embodiments, one or both of the PTD 300 and Clinician Computing Device 400 are not discrete, stand-alone devices. Instead, for example, some or all of the hardware and/or software of a Patient Computing Device 100a, 100b and a Patient Testing Device 300 may be integrated into a single device. For non-limiting illustrative purposes only, hardware, software and functionality of the Patient Computing Devices 100a, 100b and the Patient Testing Device 300 are discussed with reference to an exemplary combined device 300 of FIG. 4. Similarly, some or all of the hardware and/or software of a Clinician Computing Device 200a, 200b and a Clinician Control Device 300b may be integrated into a single device. For non-limiting illustrative purposes only, hardware, software and functionality of the Clinician Computing Devices 200a, 200b and the Clinician Control Device 400 are discussed with reference to an exemplary combined device 400 of FIG. 5.

In accordance with another aspect of the present invention, the exemplary network environment 10 further includes a Remote Testing System (RTS) 500. By way of example, the RTS 500 may be implements as a software-based process as a web/cloud-based service using conventional hardware and software and additional software providing the functionality described herein. In certain embodiments, the RTS 500 may be modified to include software in accordance with the present invention to provide hardware, software and/or functionality described herein a residing elsewhere in the exemplary network computing environment 10, or its hardware, software and/or functionality may be incorporated into one or more of the other devices described herein.

Hardware and software for enabling communication of data by such systems via such communications networks are well known in the art and beyond the scope of the present invention, and thus are not discussed in detail herein.

### Patient Computing Device

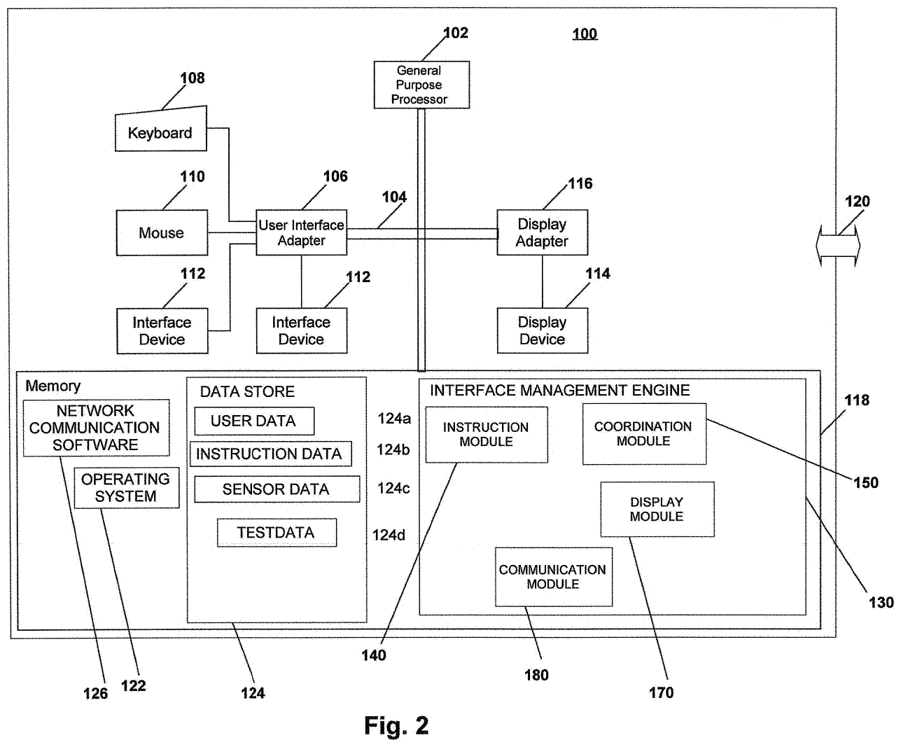

FIG. 2 is a schematic block diagram illustrating an exemplary Patient Computing Device (PCD) 100 (e.g., 100a, 100b) in accordance with an exemplary embodiment of the present invention. In certain embodiments, the PCD 100 may be a general-purpose computer including conventional computing hardware and software that is caused, e.g., under control of the RTS 500, to cause display information and/or receive inputs from a patient/user in accordance with the present invention. The exemplary PCD 100 is a special-purpose computer system that includes conventional computing hardware storing and executing both conventional software enabling operation of a general-purpose computing system, such as operating system software, network communications software, and also specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Referring again to FIG. 2, a block diagram of an exemplary PCD 100 according to some embodiments is shown. In some embodiments, the PCD 100 may, for example, execute, process, facilitate, and/or otherwise be associated with the embodiments described above.

Accordingly, the exemplary PCD 100 of FIG. 2 includes a general-purpose processor, such as a microprocessor (CPU), 102 and a bus 104 employed to connect and enable communication between the processor 102 and the components of the presentation system in accordance with known techniques. According to some embodiments, the processor 102 may be or include any type, quantity, and/or configuration of processor that is or becomes known. In some embodiments, the processor 102 may comprise multiple inter-connected processors, microprocessors, and/or micro-engines. According to some embodiments, the processor 102 (and/or the system 100 and/or other components thereof) may be supplied power via a power supply (not shown), such as a battery, an Alternating Current (AC) source, a Direct Current (DC) source, an AC/DC adapter, solar cells, and/or an inertial generator.

The exemplary PCD 100 includes a user interface adapter 106, which connects the processor 102 via the bus 104 to one or more interface devices, such as a keyboard 108, mouse 110, and/or other interface devices 112, which can be any user interface device, such as a touch sensitive screen, digitized entry pad, etc. The bus 104 also connects a display device 114, such as an LCD screen or monitor, to the processor 102 via a display adapter 116.

The bus 104 also connects the processor 102 to memory 118, which can include a hard drive, a solid-state drive, an optical drive, a diskette drive, a tape drive, etc. The memory 118 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 118 may, according to some embodiments, store one or more software components. Any or all of the exemplary instructions and data types described herein and other practicable types of data may be stored in any number, type, and/or configuration of memory systems that is or becomes known. The memory 118 may, for example, comprise one or more data tables or files, databases, table spaces, registers, and/or other storage structures. In some embodiments, multiple databases and/or storage structures (and/or multiple memory systems) may be utilized to store information associated with the system 100. According to some embodiments, the memory 118 may be incorporated into and/or otherwise coupled to the system 100 (e.g., as shown) or may simply be accessible to the system 100 (e.g., externally located and/or situated).

The PCD 100 may communicate with other computers or networks of computers, for example via a communications channel, network card, modem or transceiver (collectively, “transceiver”) 120. In some embodiments, the transceiver 120 may comprise any type or configuration of communication system that is or becomes known or practicable. The transceiver 120 may, for example, comprise a Network Interface Card (NIC), a telephonic system, a cellular network system, a router, a hub, a modem, and/or a communications port or cable. According to some embodiments, the transceiver 120 may also or alternatively be coupled to the processor 102. In some embodiments, the transceiver 120 may comprise an IR, RF, Bluetooth™, Near-Field Communication (NFC), and/or Wi-Fi® network system coupled to facilitate communications between the processor 102 and another system (not shown). The PCD 100 may be associated with such other computers in a local area network (LAN) or a wide area network (WAN), and may operate as a server in a client/server arrangement with another computer, etc. Such configurations, as well as the appropriate communications hardware and software, are known in the art.

The PCD 100 is specially-configured in accordance with the present invention. Accordingly, as shown in FIG. 2, the PCD 100 includes computer-readable, processor-executable instructions stored in the memory 118 for carrying out the methods described herein. Further, the memory 118 stores certain data, e.g., in one or more databases or other data stores 124 shown logically in FIG. 2 for illustrative purposes, without regard to any particular embodiment in one or more hardware or software components.

Further, as will be noted from FIG. 2, the PCD 100 includes, in accordance with the present invention, a data store 124 and an Interface Management Engine (IME) 130, shown schematically as stored in the memory 118, which includes a number of additional modules providing functionality in accordance with the present invention, as discussed in greater detail below. These modules may be implemented primarily by specially-configured software including microprocessor—executable instructions stored in the memory 118 of the PCD 100. Optionally, other software may be stored in the memory 118 and and/or other data may be stored in the data store 124 or memory 118. Further, the IME 130 includes one or more modules shown logically in FIG. 2 for illustrative purposes, without regard to any particular embodiment in one or more hardware or software components.

It should be noted that some of the wording and form of description herein is done to meet applicable statutory requirements. Although the terms “step”, “block”, “module”, “engine”, etc. might be used herein to connote different logical components of methods or systems employed and/or for ease of illustration, the terms should not be interpreted as implying any particular order among or between various steps herein disclosed unless and except when the order of individual steps is explicitly described, or be interpreted as implying any distinct structure separate and apart from other structures of the system.

The IME 130 is operable to store and/or receive instruction data that may be used to gather, store and/or transmit user data, to display text or other instructional cues to a patient, e.g., according to instructions received from a Clinician and/or Clinician Computing Device 200a, 200b and/or a centralized Remote Testing System 500, to communicate test or related data to a Clinician Computing Device 200a, 200b and/or to a centralized Remote Testing System 500, and in some embodiments, to gather test data resulting from the patient's interactions with a Patient Testing Device 300, as discussed in greater detail below.

In part, the PCD 100 stores User Data 124a in the data store 124, e.g., in a database cluster. The User Data 124a identifies the user and includes any relevant user-identified and user-associated data, such as contact and communication information, including information for transmitting data to and/or otherwise interfacing with the user's Patient Testing Device 300, if needed. By way of example, some or all of this information may be provided by or gathered from the user by direct input or by data communication via the network 50 or via a local communication channel (e.g., via Bluetooth) with the user's Patient Testing Device 300.

Further, the PCD 100 stores Instruction Data 124b in the data store 124. The Instruction Data 124b may be stored in the device as part of pre-configuration, e.g., with a suitable software “app”, or may be received as a transmission from another device, such as a Remote Testing System 500 or a Clinician Computing Device 200a, 200b. For example, the Instruction Data 124b may include instructions to cause display of graphical or textual instructional cues, or to reproduce audible cues, etc., to provide instructions to the user/patient for operation of the Patient Testing device 300 in accordance with requirements of a sensory and/or motor skills test.

The IME 130 includes an Instruction Module (IM) 140 that is operable to receive and/or retrieve User Data 124a and/or Instruction Data 124b from the data store 124 and to display (e.g., using the Display Module 170) or otherwise provide suitable instructions via the hardware (e.g., Display Device 114) of the PCD 100 for guiding the patient to use the Patient Testing Device 300 as desired as part of a sensory and/or motor skills test.

The IME 130 further includes a Coordination Module (CM) 150 that is operable to receive data from an external computing device, such as the Remote Testing System 500, Clinician Computing Device 200, or even the Clinician Control Device 400, to synchronize or otherwise coordinate the operation of the Clinician Computing Device 200 and/or Clinician Control Device 400 with operation of the Patient Computing Device 100 and/or the Patient Testing Device 300. For example, a clinician may operate a switch at the Clinician Control Device 400 and/or provide input at the Clinician Computing Device 200 to initiate the performance of a particular motor and/or sensory skill test, and the Coordination Module may detect, track or otherwise be informed of such event and may act in concert with the Instruction Module 140 and/or the Display Module 170 to cause corresponding instructions, from the Instruction Data 124, to be displayed, at an appropriate time, to the user to cause the user to take action to interact with the Patient Testing Device in a manner suitable for performance of the test, e.g., based on instructions displayed or otherwise delivered via the PCD 100.

In certain embodiments, the IME 130 further includes a Communication Module (COM) 180. The COM 180 is operable to communicate data via the network 50, e.g., to send and/or receiving instructions, data, test results, etc. In certain embodiments, the COM 180 is operable to communicate with a nearby Patient Testing Device 300, such as via a Bluetooth short-range wireless connection, and store Test Data 124d and communicate such data or related data to an external computing device.

Accordingly, the PCD 100 may be configured to operate as a distinct, separate device for delivering communications to a patient in coordination with actions taken at a Clinician Computing Device 200 or Clinician Control Device 400, or under common control of a Remote Testing System 500, to deliver instructions to a patient to guide the patient in taking a motor skills and/or sensory test.

### Clinician Computing Device

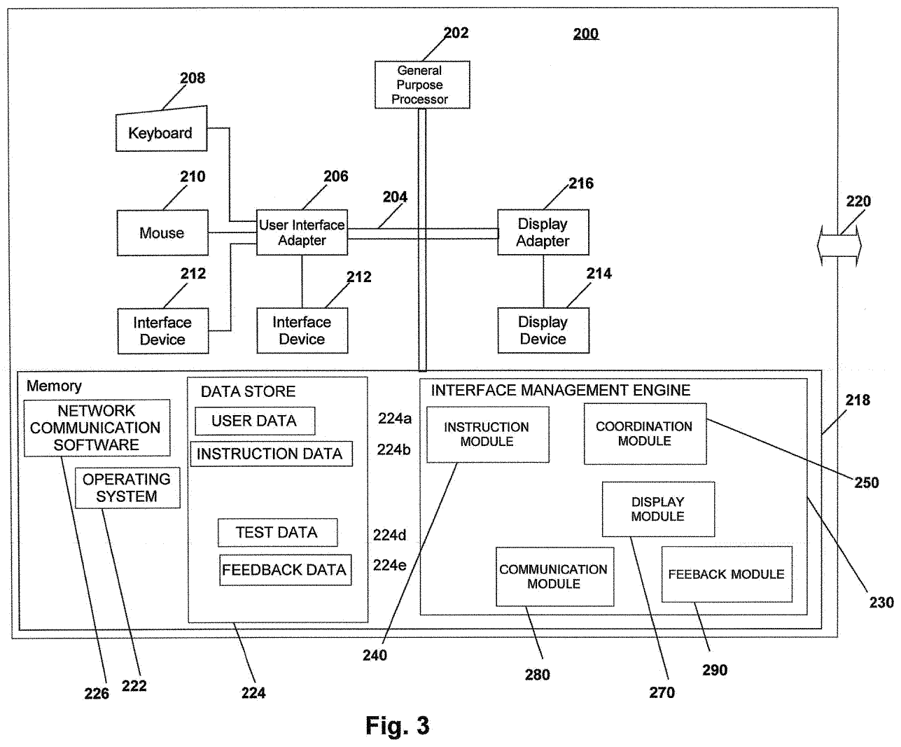

FIG. 3 is a schematic block diagram illustrating an exemplary Clinician Computing Device (CCD) 200 (e.g., 200a, 200b) in accordance with an exemplary embodiment of the present invention. In certain embodiments, the CCD 200 may be a general-purpose computer including conventional computing hardware and software that is caused, e.g., under control of the RTS 500, to cause display information and/or receive inputs from a clinician/user in accordance with the present invention. The exemplary CCD 100 is a special-purpose computer system that includes conventional computing hardware storing and executing both conventional software enabling operation of a general-purpose computing system, such as operating system software, network communications software, and also specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Referring again to FIG. 3, a block diagram of an exemplary CCD 200 according to some embodiments is shown. In some embodiments, the CCD 200 may, for example, execute, process, facilitate, and/or otherwise be associated with the embodiments described above.

Accordingly, the exemplary CCD 200 of FIG. 3 includes a general-purpose processor, such as a microprocessor (CPU), 202 and a bus 204 employed to connect and enable communication between the processor 202 and the components of the presentation system in accordance with known techniques. According to some embodiments, the processor 202 may be or include any type, quantity, and/or configuration of processor that is or becomes known. In some embodiments, the processor 202 may comprise multiple inter-connected processors, microprocessors, and/or micro-engines. According to some embodiments, the processor 202 (and/or the system 200 and/or other components thereof) may be supplied power via a power supply (not shown), such as a battery, an Alternating Current (AC) source, a Direct Current (DC) source, an AC/DC adapter, solar cells, and/or an inertial generator.

The exemplary CCD 200 includes a user interface adapter 206, which connects the processor 202 via the bus 204 to one or more interface devices, such as a keyboard 208, mouse 220, and/or other interface devices 212, which can be any user interface device, such as a touch sensitive screen, digitized entry pad, etc. The bus 204 also connects a display device 214, such as an LCD screen or monitor, to the processor 202 via a display adapter 216.

The bus 204 also connects the processor 202 to memory 218, which can include a hard drive, a solid-state drive, an optical drive, a diskette drive, a tape drive, etc. The memory 218 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 218 may, according to some embodiments, store one or more software components. Any or all of the exemplary instructions and data types described herein and other practicable types of data may be stored in any number, type, and/or configuration of memory systems that is or becomes known. The memory 218 may, for example, comprise one or more data tables or files, databases, table spaces, registers, and/or other storage structures. In some embodiments, multiple databases and/or storage structures (and/or multiple memory systems) may be utilized to store information associated with the system 200. According to some embodiments, the memory 218 may be incorporated into and/or otherwise coupled to the system 200 (e.g., as shown) or may simply be accessible to the system 200 (e.g., externally located and/or situated).

The CCD 200 may communicate with other computers or networks of computers, for example via a communications channel, network card, modem or transceiver (collectively, “transceiver”) 220. In some embodiments, the transceiver 220 may comprise any type or configuration of communication system that is or becomes known or practicable. The transceiver 220 may, for example, comprise a Network Interface Card (NIC), a telephonic system, a cellular network system, a router, a hub, a modem, and/or a communications port or cable. According to some embodiments, the transceiver 220 may also or alternatively be coupled to the processor 202. In some embodiments, the transceiver 220 may comprise an IR, RF, Bluetooth™, Near-Field Communication (NFC), and/or Wi-Fi® network system coupled to facilitate communications between the processor 202 and another system (not shown). The PCD 200 may be associated with such other computers in a local area network (LAN) or a wide area network (WAN), and may operate as a server in a client/server arrangement with another computer, etc. Such configurations, as well as the appropriate communications hardware and software, are known in the art.

The CCD 200 is specially-configured in accordance with the present invention. Accordingly, as shown in FIG. 2, the CCD 200 includes computer-readable, processor-executable instructions stored in the memory 218 for carrying out the methods described herein. Further, the memory 218 stores certain data, e.g., in one or more databases or other data stores 224 shown logically in FIG. 3 for illustrative purposes, without regard to any particular embodiment in one or more hardware or software components.

Further, as will be noted from FIG. 3, the CCD 200 includes, in accordance with the present invention, a data store 224 and an Interface Management Engine (IME) 230, shown schematically as stored in the memory 218, which includes a number of additional modules providing functionality in accordance with the present invention, as discussed in greater detail below. These modules may be implemented primarily by specially-configured software including microprocessor—executable instructions stored in the memory 218 of the CCD 200. Optionally, other software may be stored in the memory 218 and and/or other data may be stored in the data store 224 or memory 218. Further, the IME 230 includes one or more modules shown logically in FIG. 3 for illustrative purposes, without regard to any particular embodiment in one or more hardware or software components.

The IME 230 is operable to store and/or receive instruction data that may be used to gather, store and/or transmit user data, to display text or other instructional cues to a clinician, e.g., according to instructions or other data received from a Patient Computing Device 100a, 100b and/or a centralized Remote Testing System 500, to communicate instruction, test or other related data to a Patient Computing Device 100a, 100b and/or to a centralized Remote Testing System 500, and in some embodiments, to receive test data resulting from the patient's interactions with a Patient Testing Device 300, as discussed in greater detail below.

In part, the CCD 200 stores User Data 224a in the data store 224, e.g., in a database cluster. The User Data 224a identifies the patient and/or clinician user and includes any relevant user-identified and user-associated data, such as contact and communication information, including information for transmitting data to and/or otherwise interfacing with the user's Patient Computing Device 100 and/or Patient Testing Device 300, if needed. By way of example, some or all of this information may be provided by or gathered from the user by direct input or by data communication via the network 50 or via a local communication channel (e.g., via Bluetooth) with the clinician's Clinician Control Device 300.

Further, the CCD 200 stores Instruction Data 224b in the data store 224. The Instruction Data 224b may be stored in the device as part of pre-configuration, e.g., with a suitable software “app”, or may be received as a transmission from another device, such as a Remote Testing System 500 or a Patient Computing Device 100a, 100b. For example, the Instruction Data 224b may include instructions to cause display of graphical or textual instructional cues, or to reproduce audible cues, etc., to provide instructions to the clinician via the CCD 200 and/or the Clinician Control Device 300 and/or to the Patient Computing Device 100a, 100b and/or to the user/patient via the Patient Testing device 300 in accordance with requirements of a sensory and/or motor skills test.

The IME 230 includes an Instruction Module (IM) 240 that is operable to receive and/or retrieve User Data 224a and/or Instruction Data 224b from the data store 224 and to display (e.g., using the Display Module 270) or otherwise provide suitable instructions via the hardware (e.g., Display Device 214) of the CCD 200 for guiding the clinician to use the Clinician Computing Device 200 and/or the Clinician Control Device 300 and/or for guiding the patient to use the Patient Computing Device 100 and/or the Patient Testing Device 300 as desired as part of a sensory and/or motor skills test.

The IME 230 further includes a Coordination Module (CM) 250 that is operable to send data to and/or receive data from an external computing device, such as the Remote Testing System 500, Patient Computing Device 100, the Patient Testing Device 300 and/or the Clinician Control Device 400, to synchronize or otherwise coordinate the operation of the CCD 200 and/or Clinician Control Device 400 with operation of the PCD 100 and/or the Patient Testing Device 300. For example, a clinician may operate a switch at the Clinician Control Device 400 and/or provide input at the CCD 200 to initiate the performance of a particular motor and/or sensory skill test, and the Coordination Module 250 may detect, track or otherwise be informed of such event and may act in concert with the Instruction Module 240 and/or the Display Module 270 to cause corresponding instructions, e.g., from the Instruction Data 224, to be displayed, at an appropriate time, to the clinician and/or patient/user to cause the patient to take action to interact with the Patient Testing Device 300 in a manner suitable for performance of a motor or sensory skills test, e.g., based on instructions displayed or otherwise delivered via the CCD 200.

In certain embodiments, the IME 230 further includes a Communication Module (COM) 280. The COM 280 is operable to communicate data via the network 50, e.g., to send and/or receiving instructions, data, test results, etc. In certain embodiments, the COM 280 is operable to communicate with a nearby Clinician Control Device 400, such as via a Bluetooth short-range wireless connection, and store Test Data 224d and communicate such data or related data to an external computing device.

Additionally, in certain embodiments, the IME 230 further includes a Feedback Module (FM) 290. Acting in concert with the COM 280, the FM 290 is operable to receive Test Data 224 via the network 50 and to create/generate corresponding feedback data, to store such Feedback Data 224e in the data store 224, and to transmit a suitable feedback control signal to the Clinician Control Device 400 (or the CCD 200) to cause the Clinician Control Device 400 (or CCD 200) to operate to provide feedback to the clinician as a function of input provided by the patient to the Patient Testing Device 300. For example, a control signal may be used to cause display on a display device 214 of the CCD 200 to show a chart, graph, metric or other indicator of hand gripping force exerted by the patient on the joystick(s) of the PTD 300. By way of alternative example, a control signal may be used to provide haptic feedback on the Clinician Control Device 400 cause operation of the joystick(s)/levers 455 to provide mechanical/force feedback corresponding to the hand gripping force exerted by the patient on the joystick(s) of the PTD 300.

Accordingly, the CCD 200 may be configured to operate as a distinct, separate device for transmitting communications to a patient in coordination with actions taken at the Clinician Computing Device 200 or Clinician Control Device 400, or under common control of a Remote Testing System 500, to deliver instructions to a patient to guide the patient in using the PCD 100 and/or Patient Testing Device 300 in taking a motor skills and/or sensory test.

### Patient Testing Device

FIG. 4 is a schematic block diagram illustrating an exemplary and non- limiting embodiment of a Patient Testing Device (PTD) 300 in accordance with the present invention. In certain embodiments, the PTD 300 is a special-purpose device that may be used as a stand-alone device, or as a peripheral or other device associated with and used to communicate with a PCD 100 or other general-purpose computing device, such as a smartphone or tablet computing device. In such an embodiment, the PTD 300 may include primarily the housing 315 and joysticks/levers 325, touchpads and associated heating/cooling hardware and/or such other devices/mechanisms/sensors for performing the desired motor and/or sensory skills tests and such other data gathering, storage and communication components as may be desired, e.g., for communicating associated data to a PCD 100 or to another computing device. Accordingly, the PTD 300 may not include general-purpose computing hardware of a type similar to that described above in relating to the PCD 100. Rather, the PTD 300 may communication with the PCD 100 including such hardware and software.

Alternatively, however, as shown in the example of FIG. 4, the PTD 300 does include such general-purpose computing hardware and software. In such a case, the PCD 100 and the PTD 300 may be effective integrated into a single device, and it may be possible to operate the system satisfactorily without use of the PCD 100. In such a case, structure at, and functions performed, by the PCD 100 may be provided at or be performed by the PTD 300 instead of the PCD 100.

Accordingly, the exemplary PTD 300 is a special-purpose device that includes conventional computing hardware storing and executing both conventional software enabling operation of a general-purpose computing system, such as operating system software, network communications software, etc. as described above and also specially-configured hardware and computer software for configuring the PTD 300 as a special-purpose device in accordance with the present invention. The PTD 300 is caused, e.g., under control of the RTS 500, to display information and/or receive inputs from a patient/user in accordance with the present invention.

Accordingly, the exemplary PTD 300 of FIG. 4 includes a general-purpose processor, such as a microprocessor (CPU), 302 and a bus 304 employed to connect and enable communication between the processor 302 and the components of the presentation system in accordance with known techniques. According to some embodiments, the processor 302 may be or include any type, quantity, and/or configuration of processor that is or becomes known. In some embodiments, the processor 302 may comprise multiple inter-connected processors, microprocessors, and/or micro-engines. According to some embodiments, the processor 302 (and/or the system 300 and/or other components thereof) may be supplied power via a power supply (not shown), such as a battery, an Alternating Current (AC) source, a Direct Current (DC) source, an AC/DC adapter, solar cells, and/or an inertial generator.

The exemplary PTD 300 includes a user interface adapter 306, which connects the processor 302 via the bus 304 to one or more interface devices, such as a keyboard 308, mouse 310, and/or other interface devices 312, which can be any user interface device, such as a touch sensitive screen, digitized entry pad, etc. The bus 304 also connects a display device 314, such as an LCD screen or monitor, to the processor 302 via a display adapter 316.

The bus 304 also connects the processor 302 to memory 318, which can include a hard drive, a solid-state drive, an optical drive, a diskette drive, a tape drive, etc. The memory 318 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 318 may, according to some embodiments, store one or more software components. Any or all of the exemplary instructions and data types described herein and other practicable types of data may be stored in any number, type, and/or configuration of memory systems that is or becomes known. The memory 318 may, for example, comprise one or more data tables or files, databases, table spaces, registers, and/or other storage structures. In some embodiments, multiple databases and/or storage structures (and/or multiple memory systems) may be utilized to store information associated with the system 300. According to some embodiments, the memory 318 may be incorporated into and/or otherwise coupled to the system 300 (e.g., as shown) or may simply be accessible to the system 300 (e.g., externally located and/or situated).

The PTD 300 may communicate with other computers or networks of computers, for example via a communications channel, network card, modem or transceiver (collectively, “transceiver”) 320. In some embodiments, the transceiver 320 may comprise any type or configuration of communication system that is or becomes known or practicable. The transceiver 320 may, for example, comprise a Network Interface Card (NIC), a telephonic system, a cellular network system, a router, a hub, a modem, and/or a communications port or cable. According to some embodiments, the transceiver 320 may also or alternatively be coupled to the processor 302. In some embodiments, the transceiver 320 may comprise an IR, RF, Bluetooth™, Near-Field Communication (NFC), and/or Wi-Fi® network system coupled to facilitate communications between the processor 302 and another system (not shown). The PTD 300 may be associated with such other computers in a local area network (LAN) or a wide area network (WAN), and may operate as a server in a client/server arrangement with another computer, etc. Such configurations, as well as the appropriate communications hardware and software, are known in the art.

The PTD 300 is specially-configured in accordance with the present invention. Accordingly, as shown in FIG. 2, the PTD 300 includes computer-readable, processor-executable instructions stored in the memory 318 for carrying out the methods described herein. Further, the memory 318 stores certain data, e.g., in one or more databases or other data stores 324 shown logically in FIG. 4 for illustrative purposes, without regard to any particular embodiment in one or more hardware or software components.

Further, as will be noted from FIG. 4, the PTD 300 includes, in accordance with the present invention, a data store 324 and an Interface Management Engine (IME) 330, shown schematically as stored in the memory 318, which includes a number of additional modules providing functionality in accordance with the present invention, as discussed in greater detail below. These modules may be implemented primarily by specially-configured software including microprocessor—executable instructions stored in the memory 318 of the PTD 300. Optionally, other software may be stored in the memory 318 and and/or other data may be stored in the data store 324 or memory 318. Further, the IME 330 includes one or more modules shown logically in FIG. 4 for illustrative purposes, without regard to any particular embodiment in one or more hardware or software components.

The IME 330 is operable to store and/or receive instruction data that may be used to gather, store and/or transmit user data, to display text or other instructional cues to a patient, e.g., according to instructions or other data received from a CCD 200 and/or a centralized Remote Testing System 500, to communicate instruction, test or other related data to the patient and/or to a centralized Remote Testing System 500, and in some embodiments, to receive test data resulting from the patient's interactions with the PTD 300, as discussed in greater detail below.

In part, the PTD 300 stores User Data 324a in the data store 324, e.g., in a database cluster. The User Data 324a identifies the patient user and includes any relevant user-identified and user-associated data, such as contact and communication information, including information for transmitting data to and/or otherwise interfacing with the user's PTD 300. By way of example, some or all of this information may be provided by or gathered from the user by direct input or by data communication via the network 50.

Further, the PTD 300 stores Instruction Data 324b in the data store 324. The Instruction Data 324b may be stored in the device as part of pre-configuration, e.g., with a suitable software “app”, or may be received as a transmission from another device, such as a Remote Testing System 500 or a Patient Computing Device 100a, 100b. For example, the Instruction Data 324b may include instructions to cause display of graphical or textual instructional cues, or to reproduce audible cues, etc., to provide instructions to the patient via the PTD 300 and/or the Patient Computing Device 100a, 100b in accordance with requirements of a sensory and/or motor skills test.

The IME 330 includes an Instruction Module (IM) 340 that is operable to receive and/or retrieve User Data 224a and/or Instruction Data 324b from the data store 324 and to display (e.g., using the Display Module 370) or otherwise provide suitable instructions via the hardware (e.g., Display Device 314) of the PTD 300 for guiding the patient to use the PTD 300 as desired as part of a sensory and/or motor skills test.

The IME 330 further includes a Coordination Module (CM) 350 that is operable to send data to and/or receive data from an external computing device, such as the Remote Testing System 500, Patient Computing Device 100, CCD 200 and/or the Clinician Control Device 400, to synchronize or otherwise coordinate the operation of the CCD 200 and/or Clinician Control Device 400 with operation of the PTD 300. For example, a clinician may operate a switch at the Clinician Control Device 400 and/or provide input at the CCD 200 to initiate the performance of a particular motor and/or sensory skill test, and the Coordination Module 350 may detect, track or otherwise be informed of such event and may act in concert with the Instruction Module 340 and/or the Display Module 370 to cause corresponding instructions, e.g., from the Instruction Data 324, to be displayed, at an appropriate time, to the patient/user to cause the patient to take action to interact with the PTD 300 in a manner suitable for performance of a motor or sensory skills test, e.g., based on instructions displayed or otherwise delivered via the PTD 300.

In certain embodiments, the IME 330 further includes a Communication Module (COM) 380. The COM 380 is operable to communicate data via the network 50, e.g., to send and/or receiving instructions, data, test results, etc. In certain embodiments, the COM 380 is operable to communicate with a nearby PCD 100, such as via a Bluetooth short-range wireless connection, and store Test Data 224d and communicate such data or related data to an external computing device.

Additionally, in accordance with the present invention, the Interface Device 312 of FIG. 4 further includes the joysticks 325 and the touchpads 335 of FIG. 1, as well as associated hardware and/or software, such as a resistive heater and associated circuitry, refrigerants, cooling fluids and associated valves and control circuitry, etc. Further, the IME 330 further includes a Testing Device Control Module (DTCM) 395 for controlling such valves and circuitry and/or any other associated mechanisms as needed, for actuating portions of the PTD 300 in accordance with a desired motor or sensory skills test. Additionally, the IME 330 further includes a Data Acquisition Module (DAM) 360 for gathering data from the joysticks 325, touchpads 335 and any other associated sensors or mechanisms as desired in accordance with a desired motor or sensory skills test. The DAM 360 also acts to store acquired data as Sensor Data 324c in the data store 324 and to interpret the Sensor Data 324c and create and store associated Test Data 324d.

Acting in concert with the DAM 360, the COM 380 is operable to transmit associated Sensor Data 324c and/or Test Data 324d via the network 50 and to transmit a suitable feedback control signal to the Clinician Control Device 400 (or the CCD 200) to cause the Clinician Control Device 400 (or CCD 200) to operate to provide feedback to the clinician as a function of input provided by the patient to the PTD 300. For example, a control signal may be used to cause display on a display device 314 of the CCD 200 to show a chart, graph, metric or other indicator of hand gripping force exerted by the patient on the joystick(s) of the PTD 300. By way of alternative example, a control signal may be used to provide haptic feedback on the Clinician Control Device 400 cause operation of the joystick(s)/levers 455 to provide mechanical/force feedback corresponding to the hand gripping force exerted by the patient on the joystick(s) of the PTD 300.

Accordingly, the PTD 300 may be configured to operate as a device separate from the PCD 100, or as a single integrated device with the PCD 100's functionality, for operation in coordination with actions taken at the Clinician Computing Device 200 or Clinician Control Device 400, or under common control of a Remote Testing System 500, to deliver instructions to a patient to guide the patient in using the PCD 100 and/or PTD 300 in taking a motor skills and/or sensory test.

### Clinician Control Device

FIG. 5 is a schematic block diagram illustrating an exemplary and non-limiting embodiment of a Clinician Control Device (ClinConDev) 400 in accordance with the present invention. In certain embodiments, the ClinConDev 400 is a special-purpose device that may be used as a stand-alone device, or as a peripheral or other device associated with and used to communicate with a CCD 200 or other general-purpose computing device, such as a smartphone or tablet computing device. In such an embodiment, the ClinConDev 400 may include primarily the housing 415 and switches 445 (e.g., for controlling heating/cooling hardware and/or such other devices/mechanisms/sensors of the PTD 300 for performing the desired motor and/or sensory skills tests), feedback joysticks/levers 455, and such other data gathering, storage and communication components as may be desired for communicating associated data to another computing device. Accordingly, the ClinConDev 400 may not include general-purpose computing hardware of a type similar to that described above in relating to the CCD 200. Rather, the ClinConDev 400 may communicate with a CCD 200 that includes such hardware and software.

Alternatively, however, as shown in the example of FIG. 5, the ClinConDev 400 does include such general-purpose computing hardware and software. In such a case, the CCD 200 and the ClinConDev 400 may be effectively integrated into a single device, and it may be possible to operate the system satisfactorily without use of the CCD 200. In such a case, structure at, and functions performed by, the CCD 200 may be provided at or be performed by the ClinConDev 400 instead of the CCD 200.

Accordingly, the exemplary ClinConDev 400 is a special-purpose device that may include conventional computing hardware storing and executing both conventional software enabling operation of a general-purpose computing system, such as operating system software, network communications software, etc. as described above and also specially-configured hardware and computer software for configuring the ClinConDev 400 as a special-purpose device in accordance with the present invention. The ClinConDev 400 is caused, e.g., under control of the RTS 500, to display information and/or receive inputs from a clinician/user in accordance with the present invention.

Accordingly, the exemplary ClinConDev 400 of FIG. 5 includes a general-purpose processor, such as a microprocessor (CPU), 402 and a bus 404 employed to connect and enable communication between the processor 402 and the components of the presentation system in accordance with known techniques. According to some embodiments, the processor 402 may be or include any type, quantity, and/or configuration of processor that is or becomes known. In some embodiments, the processor 402 may comprise multiple inter-connected processors, microprocessors, and/or micro-engines. According to some embodiments, the processor 402 (and/or the system 400 and/or other components thereof) may be supplied power via a power supply (not shown), such as a battery, an Alternating Current (AC) source, a Direct Current (DC) source, an AC/DC adapter, solar cells, and/or an inertial generator.

The exemplary ClinConDev 400 includes a user interface adapter 406, which connects the processor 402 via the bus 404 to one or more interface devices, such as a keyboard 408, mouse 410, and/or other interface devices 412, which can be any user interface device, such as a touch sensitive screen, digitized entry pad, etc. The bus 404 also connects a display device 414, such as an LCD screen or monitor, to the processor 402 via a display adapter 416.

The bus 404 also connects the processor 402 to memory 418, which can include a hard drive, a solid-state drive, an optical drive, a diskette drive, a tape drive, etc. The memory 418 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 418 may, according to some embodiments, store one or more software components. Any or all of the exemplary instructions and data types described herein and other practicable types of data may be stored in any number, type, and/or configuration of memory systems that is or becomes known. The memory 418 may, for example, comprise one or more data tables or files, databases, table spaces, registers, and/or other storage structures. In some embodiments, multiple databases and/or storage structures (and/or multiple memory systems) may be utilized to store information associated with the system 400. According to some embodiments, the memory 418 may be incorporated into and/or otherwise coupled to the system 400 (e.g., as shown) or may simply be accessible to the system 400 (e.g., externally located and/or situated).

The ClinConDev 400 may communicate with other computers or networks of computers, for example via a communications channel, network card, modem or transceiver (collectively, “transceiver”) 420. In some embodiments, the transceiver 420 may comprise any type or configuration of communication system that is or becomes known or practicable. The transceiver 420 may, for example, comprise a Network Interface Card (NIC), a telephonic system, a cellular network system, a router, a hub, a modem, and/or a communications port or cable. According to some embodiments, the transceiver 420 may also or alternatively be coupled to the processor 402. In some embodiments, the transceiver 420 may comprise an IR, RF, Bluetooth™, Near-Field Communication (NFC), and/or Wi-Fi® network system coupled to facilitate communications between the processor 402 and another system (not shown). The PTD 400 may be associated with such other computers in a local area network (LAN) or a wide area network (WAN), and may operate as a server in a client/server arrangement with another computer, etc. Such configurations, as well as the appropriate communications hardware and software, are known in the art.

The ClinConDev 400 is specially-configured in accordance with the present invention. Accordingly, as shown in FIG. 5, the ClinConDev 400 includes computer-readable, processor-executable instructions stored in the memory 418 for carrying out the methods described herein. Further, the memory 418 stores certain data, e.g., in one or more databases or other data stores 424 shown logically in FIG. 5 for illustrative purposes, without regard to any particular embodiment in one or more hardware or software components.

Further, as will be noted from FIG. 4, the ClinConDev 400 includes, in accordance with the present invention, a data store 424 and an Interface Management Engine (IME) 430, shown schematically as stored in the memory 418, which includes a number of additional modules providing functionality in accordance with the present invention, as discussed in greater detail below. These modules may be implemented primarily by specially-configured software including microprocessor—executable instructions stored in the memory 418 of the ClinConDev 400. Optionally, other software may be stored in the memory 418 and and/or other data may be stored in the data store 424 or memory 418. Further, the IME 430 includes one or more modules shown logically in FIG. 5 for illustrative purposes, without regard to any particular embodiment in one or more hardware or software components.

The IME 430 is operable to store and/or receive instruction data that may be used to gather, store and/or transmit user data, to display text or other instructional cues to a patient, e.g., according to instructions or other data received from a CCD 200 and/or a centralized Remote Testing System 500, to communicate instruction, test or other related data to the patient and/or to a centralized Remote Testing System 500, and in some embodiments, to receive test data resulting from the patient's interactions with the ClinConDev 400, as discussed in greater detail below.

In part, the ClinConDev 400 stores User Data 424a in the data store 424, e.g., in a database cluster. The User Data 424a identifies the clinician and/or patient user and includes any relevant user-identified and user-associated data, such as contact and communication information, including information for transmitting data to and/or otherwise interfacing with the user's CCD 200. By way of example, some or all of this information may be provided by or gathered from the user by direct input or by data communication via the network 50.

Further, the ClinConDev 400 stores Instruction Data 424b in the data store 424. The Instruction Data 424b may be stored in the device as part of pre-configuration, e.g., with a suitable software “app”, or may be received as a transmission from another device, such as a Remote Testing System 500. For example, the Instruction Data 424b may include instructions to cause display of graphical or textual instructional cues, or to reproduce audible cues, etc., to provide instructions to the clinician via the ClinConDev 400 and/or the CCD 200 in accordance with actions of the patient at the PCD 100 and/or PTD 300 and/or requirements of a sensory and/or motor skills test.

The IME 430 includes an Instruction Module (IM) 445 that is operable to receive and/or retrieve User Data 224a and/or Instruction Data 424b from the data store 424 and to display (e.g., using the Display Module 470) or otherwise provide suitable instructions via the hardware (e.g., Display Device 414) of the ClinConDev 400 for guiding the clinician to use the ClinConDev 400 as desired as part of a sensory and/or motor skills test.

The IME 430 further includes a Coordination Module (CM) 455 that is operable to send data to and/or receive data from an external computing device, such as the Remote Testing System 500, PCD 100, CCD 200 and/or the PTD 300, to synchronize or otherwise coordinate the operation of the ClinConDev 400 and/or CCD 200 with the operation of the PCD 100 and/or the PTD 300. For example, a clinician may operate a switch at the ClinConDev 400 and/or provide input at the CCD 200 to initiate the performance of a particular motor and/or sensory skill test, and the Coordination Module 455 may detect, track or otherwise be informed of such event and may act in concert with the Instruction Module 445 and/or the Display Module 470 to cause corresponding instructions, e.g., from the Instruction Data 424b, to be displayed, at an appropriate time, to the patient/user to cause the patient to take action to interact with the PCD 100 and/or the PTD 300 in a manner suitable for performance of a motor or sensory skills test, e.g., based on instructions displayed or otherwise delivered via the ClinConDev 400.

In certain embodiments, the IME 430 further includes a Communication Module (COM) 480. The COM 480 is operable to communicate data via the network 50, e.g., to send and/or receiving instructions, data, test results, etc. In certain embodiments, the COM 480 is operable to communicate with a nearby CCD 200, such as via a Bluetooth short-range wireless connection, and store Test Data 224d and communicate such data or related data to an external computing device.

Additionally, in accordance with the present invention, the Interface Device 412 of FIG. 5 further includes switches 445 that may be user-manipulable in fashions relevant to testing of the patient's motor and/or sensory functions at the Patient Testing Device 300. By way of example, one or more switches 445 may be provided to selectively cause a touchpad 330 to be heated or cooled at the Patient Testing Device 300.

Additionally, in accordance with the present invention, the Interface Device 412 of FIG. 5 further includes joysticks or levers 455 that may be user-manipulable in fashions relevant to testing of the patient's motor and/or sensory functions and/or may be driven to provide haptic feedback to a user at the ClinConDev 400 based on patient input at the PTD 300, as well as associated hardware and/or software, such as a electric motors, actuators, etc. and control circuitry. Accordingly, movement and/or pressure exerted by a patient on the joysticks 325 of the PTD 300 may be effectively replicated by driving/actuation of the joysticks/levers, a clamp, or other device at the ClinConDev 400, so that the clinician at the ClinConDev 400 can effectively feel/experience the actions of the patient at the PTD 300.

Additionally, the IME 430 further includes a Feedback Module (FM) 490. Acting in concert with the COM 480, the FM 490 is operable to receive Sensor Data and/or Test Data 224 via the network 50 and to create/generate corresponding feedback data, to store such Feedback Data 424e in the data store 424, and to transmit a suitable feedback control signal to the ClinConDev 400 (or the CCD 200) to cause the ClinConDev 400 (or CCD 200) to operate to provide feedback to the clinician as a function of input provided by the patient to the PTD 300. For example, a control signal may be used to cause display on a display device 414 of the ClinConDev 400 to show a chart, graph, metric or other indicator of hand gripping force exerted by the patient on the joystick(s) of the PTD 300. By way of alternative example, a control signal may be used to provide haptic feedback on the ClinConDev 400 by causing operation of the joystick(s)/levers 455 to provide mechanical/force feedback corresponding to the hand gripping force exerted by the patient on the joystick(s) of the PTD 300.

Accordingly, the ClinConDev 400 may be configured to operate as a device separate from the CCD 200, or as a single integrated device with the CCD 200's functionality, for operation in coordination with actions taken at the PCT 100 PTD 300, or under common control of a Remote Testing System 500, to deliver instructions to a patient to guide the patient in using the PCD 100 and/or PTD 300 in taking a motor skills and/or sensory test.

### Remote Testing System

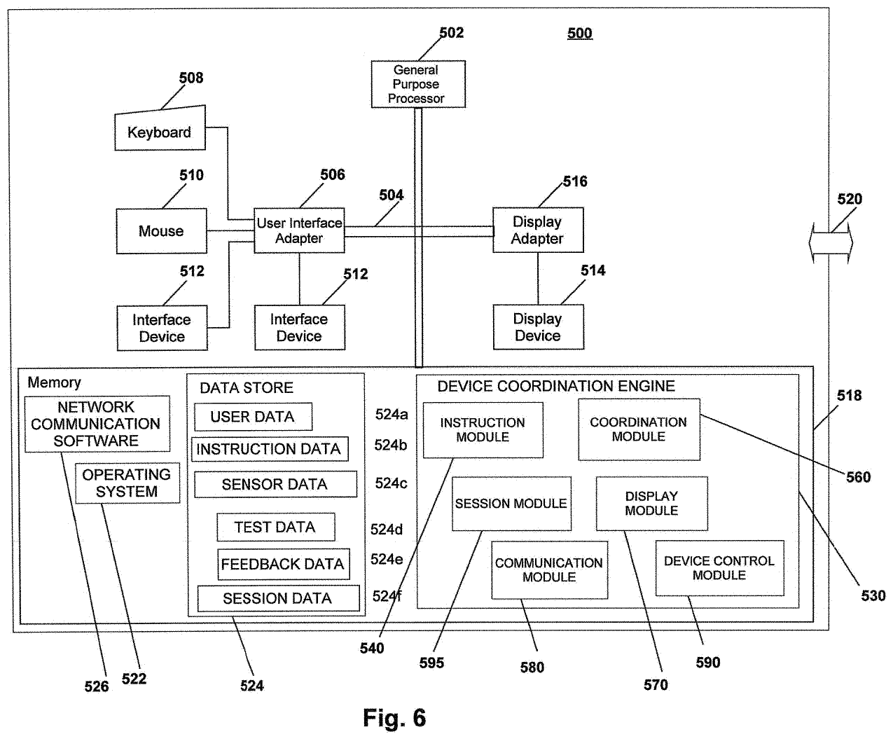

FIG. 6 is a schematic block diagram illustrating an exemplary and non-limiting embodiment of a computerized Remote Testing System (RTS) 500 in accordance with the present invention. The RTS 500 is a special-purpose computer system that includes conventional computing hardware storing and executing both conventional software enabling operation of a general-purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Referring again to FIG. 6, there is illustrated a block diagram of an exemplary RTS 500 according to some embodiments is shown. In some embodiments, the RTS 500 may, for example, execute, process, facilitate, and/or otherwise be associated with the embodiments described above.

Accordingly, the exemplary RTS 500 of FIG. 6 includes a general-purpose processor, such as a microprocessor (CPU), 502 and a bus 504 employed to connect and enable communication between the processor 502 and the components of the presentation system in accordance with known techniques. According to some embodiments, the processor 502 may be or include any type, quantity, and/or configuration of processor that is or becomes known. In some embodiments, the processor 502 may comprise multiple inter-connected processors, microprocessors, and/or micro-engines. According to some embodiments, the processor 502 (and/or the system 500 and/or other components thereof) may be supplied power via a power supply (not shown), such as a battery, an Alternating Current (AC) source, a Direct Current (DC) source, an AC/DC adapter, solar cells, and/or an inertial generator. In the case that the system 500 comprises a server, such as a blade server, necessary power may be supplied via a standard AC outlet, power strip, surge protector, and/or Uninterruptible Power Supply (UPS) system.

The exemplary RTS 500 includes a user interface adapter 506, which connects the processor 502 via the bus 504 to one or more interface devices, such as a keyboard 508, mouse 510, and/or other interface devices 512, which can be any user interface device, such as a touch sensitive screen, digitized entry pad, etc. The bus 504 also connects a display device 514, such as an LCD screen or monitor, to the processor 502 via a display adapter 516.

The bus 504 also connects the processor 502 to memory 518, which can include a hard drive, a solid-state drive, an optical drive, a diskette drive, a tape drive, etc. The memory 518 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM).

The memory 518 may, according to some embodiments, store one or more software components. Any or all of the exemplary instructions and data types described herein and other practicable types of data may be stored in any number, type, and/or configuration of memory systems that is or becomes known. The memory 518 may, for example, comprise one or more data tables or files, databases, table spaces, registers, and/or other storage structures. In some embodiments, multiple databases and/or storage structures (and/or multiple memory systems) may be utilized to store information associated with the system 500. According to some embodiments, the memory 518 may be incorporated into and/or otherwise coupled to the system 500 (e.g., as shown) or may simply be accessible to the system 500 (e.g., externally located and/or situated).

The RTS 500 may communicate with other computers or networks of computers, for example via a communications channel, network card, modem or transceiver (collectively, “transceiver”) 520. In some embodiments, the transceiver 520 may comprise any type or configuration of communication system that is or becomes known or practicable. The transceiver 520 may, for example, comprise a Network Interface Card (NIC), a telephonic system, a cellular network system, a router, a hub, a modem, and/or a communications port or cable. According to some embodiments, the transceiver 520 may also or alternatively be coupled to the processor 502. In some embodiments, the transceiver 520 may comprise an IR, RF, Bluetooth™, Near-Field Communication (NFC), and/or Wi-Fi® network system coupled to facilitate communications between the processor 502 and another system (not shown). The RTS 500 may be associated with such other computers in a local area network (LAN) or a wide area network (WAN), and may operate as a server in a client/server arrangement with another computer, etc. Such configurations, as well as the appropriate communications hardware and software, are known in the art.

The RTS 500 is specially-configured in accordance with the present invention. Accordingly, as shown in FIG. 6, the RTS 500 includes computer-readable, processor-executable instructions stored in the memory 518 for carrying out the methods described herein. Further, the memory 518 stores certain data, e.g., in one or more databases or other data stores 524 shown logically in FIG. 6 for illustrative purposes, without regard to any particular embodiment in one or more hardware or software components.

Further, as will be noted from FIG. 6, the RTS 500 includes, in accordance with the present invention, a Device Coordination Engine (DCE) 530, shown schematically as stored in the memory 518, which includes a number of additional modules providing functionality in accordance with the present invention, as discussed in greater detail below. These modules may be implemented primarily by specially-configured software including microprocessor—executable instructions stored in the memory 518 of the RTS 500. Optionally, other software may be stored in the memory 518 and and/or other data may be stored in the data store 524 or memory 518. Further, the DCE 530 includes one or more modules shown logically in FIG. 6 for illustrative purposes, without regard to any particular embodiment in one or more hardware or software components.

As shown in FIG. 6, the RTS 500 includes a data store 524 and a Device Coordination Engine (DCE) 530 in accordance with the present invention. The DCE is operable to receive User Data 524a, Instruction Data 524b Sensor Data 524c, Test Data 524d and/or Feedback Data 524e that may be used to coordinate communications among the PCD 100, PTD 300, CCD 200 and/or ClinConDev 400 to facilitate a clinician's control of, and/or a patient's participation in, a motor and/or sensory skills test, as discussed in greater detail below. The RTS 500 may store data in the data store 524, e.g., in a database cluster, and may receive this data from one of more of the PCD 100, PTD 300, CCD 200 and/or ClinConDev 400 in accordance with actions of the patient at the PCD 100 and/or PTD 300, the clinician at the CCD 200 and/or the ClinConDev 400 and/or requirements of a sensory and/or motor skills test.

The DCE 520 includes an Instruction Module 540, Display Module 670, Communication Module 680 and Device Control Module 690 (operable to control the PTD 300 and/or the ClinConDev 400) similar to those described above. In certain embodiments, the Device Control Module 690 may be operable to receive raw Sensor Data (e.g., acquired by the DAM 360 of a PTD 300) and to create/generate associated Feedback Data as a function of such Sensor Data, such that such Feedback Data can be provided to the CCD 200 and/or ClinConDev 400. In other words, the DCM 590 may process and/or analyze Sensor data to generate appropriate Feedback Data 524e to be provided to the clinician via the CCD 200 and/or the ClinConDev 400.

Notably, the DCE 530 further includes a Coordination Module (CM) 560 similar to that described above. In certain embodiments, the CM 560 of the DCE 530 may provide the ultimate control for coordinating actions at the PCD 100, PTD 300, CCD 200 and ClinConDev 400 with the assistance of their respective Coordination Module such that displays, device actuations, etc. at each device are coordination in time and in functionality so that such devices may act in tandem as needed in support of a motor and/or sensory skills test. In certain embodiments, the RTE 500 may act as an intermediary between such devices, issuing control communications to the various devices and/or relaying communications among such devices, e.g., in real time.

Additionally, the DCE 530 further includes a Session Module 560 similar to that described above. In certain embodiments, the Session Module 560 is operable to manage not only synchronous sessions, e.g., session in which the patient and clinician are participating concurrently in a real-time motor and/or sensory skills test via their respective devices, but also asynchronous session, e.g., sessions in which the patient and clinician are not participating concurrently in a real-time motor and/or sensory skills test via their respective devices. For example, in an asynchronous session, the Session Module 595 and Coordination Module 560 may cooperate to facilitate communications with the patient (e.g., including display of instructions) at the PCD 100 and/or the PTD 300 at a first time in a first session, and may also cooperate to facilitate communications with the clinician (e.g., including display of instructions, display of feedback via the CCD 200 and/or the ClinConDev 400, providing of feedback via the ClinConDev 400, etc.) at a second time in a second session after the first session. Accordingly, for example, feedback data forwarded to the clinician end allows for the ClinConDev 400 to provide real-time feedback to the clinician e.g., as if the user were squeezing the clinician's hand in the form of a display via the CCD 200 and/or ClinConDev 400 and/or via haptic feedback delivered via the ClinConDev 400 not only during a single patient/clinician session, but also at a later time. Accordingly, for example, a patient user can perform motor and sensory function tests without the participation of a clinician at the same time in the same session. More particularly, the data associated with the patient's interaction with the PTD 300 and/or the PCD 100 is stored, e.g., at the RTS 500 (or the CCD 200 or ClinConDev 400) for future use and analysis. Additionally, the Session Module 595, e.g., in cooperation with the CM 560 and the COM 580, allows the clinician to view (via a display on the CCD 200 and/or ClinConDev 400) a menu and select a specific date/time a patient user performed a test, and then to choose to receive the associate data (e.g., from data stored at the RTS 500 or another device), to view or otherwise receive feedback, including haptic feedback, at the CCD 200 and/or the ClinConDev 400 according to the patient's performance during an earlier session.

In certain embodiments, the Session Module 595, acting in concert with the CM 560 and/or COM 580, may include artificial intelligence or logic that is used to notify the patient/user when to engage with the PCD 100, PTD 300 and/or a clinician for next steps. For example, the SM 595 may prompt the patient user to perform another test 1 week or 1 month after an initial session, or otherwise as determined by the system, e.g., to examine a trend in patient motor and/or sensor skills.

### System Operation

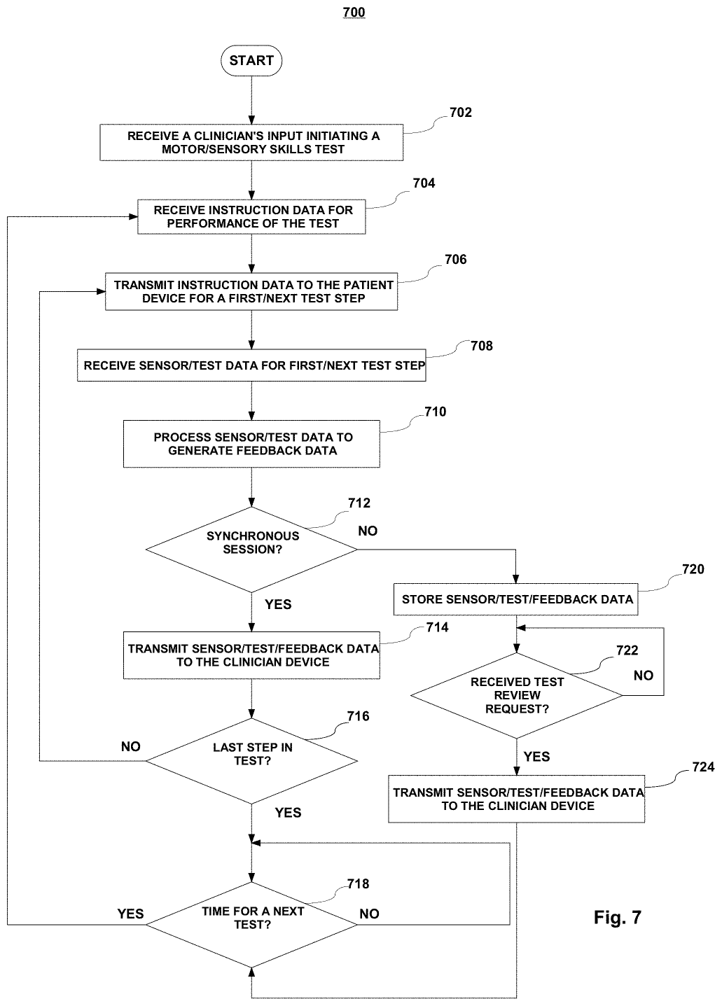

FIG. 7 is a flow diagram 700 illustrating operation of the system in accordance with an exemplary and non-limited embodiment of the present invention. Exemplary operation of the exemplary system of FIGS. 1-6 is discussed below with reference to the flow diagram 700 of FIG. 7. It should be appreciated that an exemplary method of operation in accordance with an exemplary embodiment of the present invention. For illustrative purposes, this exemplary embodiment involves the RTS 500 acting as an intermediate between patient devices and clinician devices. As noted above, in other embodiments, some or all of the functionality described with respect to the exemplary system of FIG. 1 may be performed by other devices within the system. 402. Accordingly, it should be appreciated that the exemplary embodiment described above is for illustrative purposes only, and non-limiting.

Referring now to FIG. 7, the exemplary method begins with receiving of a clinician's input initiating a motor and/or sensory skills test, as shown at 702. In certain embodiments, this may involve receiving at the RTS 500 of a data communication or control signal transmitted via the network 50 from a CCD 200 and/or a ClinConDev 400, or in response to clinician input at one of those devices. In other embodiments, this may involve data communication or exchange among components contained within a single device. For example, this may involve a data communication to initiate a motor and sensory skills test involving manipulation of joysticks and touching of heated and cooled touchpads on the PTD 300.

Next, the exemplary method involves receiving of instruction data for performance of the associated test, as shown at 704. In certain embodiments, this may involve receipt at the RTS 500 of a data communication or control signal transmitted via the network 50 from a CCD 200 and/or a ClinConDev 400, or in response to clinician input at one of those devices. In other embodiments, this may involve retrieval of Instruction Data 224b from a data store or another device, or from a data stored within/associated with a single device.

Next, the exemplary method involves transmitting instruction data for performance of the associated test, as shown at 706. In certain embodiments, this may involve receipt by and transmission from the RTS 500 of a data communication or control signal via the network 50 to a PCD 100 and/or a PTD 300, e.g., form the CCD 200 or the ClinConDev 400. In other embodiments, this may involve retrieval of Instruction Data from a data store within/associated with a single device, e.g., the RTS 500, the PCD 100 or the PTD 300.

Next, the exemplary method involves transmitting instruction data for performance of the associated test, as shown at 706. In certain embodiments, this may involve receipt by (e.g., from the CCD 200 or the ClinConDev 400) and transmission from the RTS 500 of a data communication or control signal via the network 50 to a PCD 100 and/or a PTD 300. In other embodiments, this may involve retrieval of Instruction Data from a data store within/associated with a single device, e.g., the RTS 500, the PCD 100 or the PTD 300. For example, this may involve receipt of instruction data for display of instructions via the PCD 100 or PTD 300 providing instructions for the patient user to grasp one or more of the joysticks 325 and manipulate them in a defined fashion (e.g., to push and/or pull the joysticks separately with each hand and/or together with one hand), according to the test to be performed.

Next, the exemplary method involves receiving sensor data (e.g., raw data from the sensors) or test data (e.g., interpreted sensor data and/or test results determined as a function of sensor data) for a first (or next) step of the motor and/or sensor skills test, as shown at 708. In certain embodiments, this may involve receipt (e.g., from the PCD 100 and/or the PTD 300) of a data communication or control signal via the network 50 by the RTS 500. In other embodiments, this may involve receipt of such data from a data store or via other data exchange within/associated with a single device, e.g., the RTS 500, the PCD 100 or the PTD 300. For example, this may involve receipt of raw sensor data captured by a Data Acquisition Module 360 of the PTD 300 as a result of manipulation of the joysticks 325 by the patient in a prescribed manner.

Next, the exemplary method involves processing sensor data (e.g., raw data from the sensors) or test data (e.g., interpreted sensor data and/or test results determined as a function of sensor data) to generate feedback data, as shown at 710. In certain embodiments, this may involve processing, analyzing and/or interpreting of the sensor data and/or test result data. By way of example, this may be performed at the PCD 100 and/or the PTD 300 (e.g., by the Testing Device Control Module 290), or at the CCD 200 and/or the ClinConDev 400 (e.g., by the Feedback Module 290, 490), or by the RTS 500 (e.g., by the Device Control Module 590). This may involve receipt of such data from a data store or via other data exchange within/associated with a single device, or may involve receipt of data from another device and processing of that data to create the feedback data. The feedback data is usable by the CCD 200 and/or the ClinConDev 400 to control the device to provide textual, graphical, haptic or other feedback to the clinician via the CCD 200 and/or the ClinConDev 400. For example, the Feedback Data may be configured to cause display of a message via the CCD 200 (e.g., to display numeric values or a chart of patient grip force according to the patient's manipulation of the joysticks 324 at the PTD 300) or to cause driving of the joysticks/levers 455 at the ClinConDev 400 so that the clinician may grasp the levers and feel haptic feedback at the ClinConDev 400 reflecting the patient grip force according to the patient's manipulation of the joysticks 325 at the PTD 300.

Next, it is determined if the session is a synchronous session, as shown at 712. For example, this may be determined by the Session Module 595 of the RTS 500. If the session is a synchronous session (meaning a clinician and patient are engaged in a test session with each other concurrently), then the sensor data, test data and/or feedback data may be transmitted to the clinician's device (e.g., CCD 200 and/or ClinConDev 400) at that time, as shown at 714. This may be determined by the Communication Module 580.

It may then be determined if the completed step of the motor/sensory test is the last step of the test, as shown at 716. This may be determined by the Instruction Module 540 and/or Coordination Module 570. If it is not the last step, then method flow returns to 706, and the next instruction for the next step (e.g., touching heated/cooled touchpads 335 of the PTD 300) may be displayed/performed, etc. This continues until all steps of the prescribed tests have been completed, at which point it is determined if it is time for a next test, as shown at 718.

If, however, it is determined at 712 that it is not a synchronous session (meaning that a clinician and patient are not engaged in a test session with each other concurrently), then the sensor data, test data and/or feedback data may be stored for later reference, e.g., in the data store 524 of the RTS 500. This may be determined by the Session Module 595. It is then determined whether a test review request has been received, e.g., from a clinician via the CCD 200 and/or ClinConDev 400, as shown at 722. This may be determined by the Instruction Module 540 and/or Coordination Module 570. When it is determined that that a test review request has been received, then the sensor data, test data and/or feedback data may be transmitted to the clinician's device (e.g., CCD 200 and/or ClinConDev 400) at that time, as shown at 724. It is then determined if it is time for a next test, as shown at 718.

When it is determined that it is time for a next test at 718, method flow continues to 704, where instruction data is received for performance of next test. This may be determined by the Session Module 595, acting in concert with one or more of the Instruction Module 540, the Coordination Module 560 and/or Communication Module 580. In certain embodiments, this may include the use of artificial intelligence or logic that is used to notify the patient/user when to engage with the PCD 100, PTD 300 and/or a clinician for next steps. For example, the SM 595 may prompt the patient user to perform another test 1 week or 1 month after an initial session, or otherwise as determined by the system, e.g., to examine a trend in patient motor and/or sensor skills. Alternatively, this may be determined according to input provided by a clinician at one of the CCD 200 and/or the ClinConDev 400 to initiate a next test.

Accordingly, the present invention allows a clinician (physician, nurse, etc.) to remotely conduct a motor function and sensory examination by having the patient (user) in a remote location position the patient's hands (or other body parts) on a device that contains multiple joysticks and/or touchpoints. The joysticks allow the user to push and pull the patient's hands in different directions, which sends signals to the clinician end of the device and delivers feedback on how well the user is performing the motor and/or sensory skills function as part of the test. The data forwarded to the clinician's device allows for the system to provide real-time feedback to the clinician as if the user were squeezing the clinician's hand or otherwise directly interacting with the clinician, in the form of a numeric value, chart, graph, etc. with the option for the clinician to haptically feel exerted force corresponding to the force exerted by the patient, either during a concurrent session with the user, or at a later time after which the patient has interacted with the system. Additionally, the patient user can perform motor and sensory function tests without the clinician participating in a session. Further, since the data is stored for future use and analysis, the clinician can select a specific date/time a user performed a test, and then receive data, including haptic feedback from the device, relative to how it was recorded by the user.

Accordingly remote and asynchronous motor function and sensory skills examination is enabled, optionally in combination with a parallel virtual (e.g., text, video, chat, voice call, etc.) communication channel. The present invention can also be employed to provide a stand-alone product for the patient user to monitor the patient's own motor and sensory functions and track progress to report/discuss with clinicians at a later date, without a specific need for a clinician device.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A system for remote motor and sensory function testing comprising:
a memory operatively comprising a non-transitory data processor-readable medium;
a data processor operatively connected to the memory; and
user interface management instructions embodied in data processor-executable code stored in the memory, said user interface management instructions being executable by the data processor to provide an interface management engine configured to:
cause display of instructions for performance of at least one of a motor function test and a sensory test at a remotely located computing device, the instructions comprising instructions for a test involving interacting with a patient testing device by physical contact with the patient testing device, the patient testing device comprising at least one sensor for gathering sensor data as a result of the physical contact;
receive sensor data corresponding to the physical contact with the patient testing device;
process the sensor data to generate feedback data, the feedback data indicating a patient's interaction with the patient testing device; and
transmit the feedback data via a communications network.

2. The system of claim 1, wherein the remotely located computing device comprises a patient computing device separate from the patient testing device.

3. The system of claim 1, wherein the remotely located computing device comprises the patient testing device, the patient testing device comprising:
at least one of a joystick and a temperature-controlled touchpad.

4. The system of claim 3, wherein the remotely located computing device comprises a clinician computing device separate from the patient testing device.

5. The system of claim 1, wherein the remotely located computing device comprises a clinician control device separate from the patient testing device, said clinician control device comprising at least one joystick, and wherein said clinician control device is operable to provide haptic feedback via the at least one joystick corresponding to input provided via the joystick at the patient testing device.

6. The system of claim 1, further comprising instructions to:
store the feedback data in the memory;
wherein said instructions to transmit the feedback data via the communications network involves retrieving the feedback data from the memory, and wherein the said instructions cause the transmitting in response to a request for transmission of the feedback data at a time after completion of the at least one of the motor function test and the sensory test.

7. The system of claim 1, wherein said instructions for a test involving interacting with a patient testing device by physical contact with the patient testing device comprise instructions to display instructions at a patient computing device for using a patient testing device and at a clinician computing device for using a clinician control device, said causing of display of instructions at the patient computing device and the clinician computing device being coordinated in concert in support of performance of the at least one of the motor function test and the sensory test.

8. The system of claim 1, wherein said instructions for causing display of instructions for performance of the at least one of the motor function test and the sensory test comprise causing display of first instruction data at a first time for a first test and causing display of second instruction data at a second time for a second test, and wherein the causing of display of second instruction data at the second time for the second test is a result of sending of the second instruction data at the second time as determined automatedly according to data gathered during the first test.

9. A computer-implemented method for controlling a display of a computerized device to perform remote motor and sensory function testing, the computerized device comprising a memory comprising a non-transitory data processor- readable medium, a data processor operatively connected to the memory, the display, and user interface management instructions embodied in data processor-executable code stored in the memory and executable by the data processor to provide an interface management engine, the method comprising:
receiving instruction data for performance of at least one of a motor function test and a sensory test;
displaying instructions for performing the at least one of a motor function test and a sensory test, the instructions comprising instructions to interact with a patient testing device by physical contact with the patient testing device, the patient testing device comprising at least one sensor for gathering sensor data as a result of the physical contact;
gathering sensor data corresponding to the physical contact with the patient testing device;
processing the sensor data to generate feedback data, the feedback data indicating the patient's interaction with the patient testing device and being operable to control a clinician control device to provide feedback corresponding to the patient's interaction; and
transmitting the feedback data via a communications network.

10. The method of claim 9, further comprising:
storing the feedback data in the memory;
wherein said transmitting the feedback data via the communications network involves retrieving the feedback data from the memory, and wherein said transmitting is performed in response to a request for transmission of the feedback data at a time after completion of the at least one of the motor function test and the sensory test.

11. The method of claim 9, wherein said transmitting the feedback data via the communications network comprises transmitting the feedback data to one of a clinician computing device and the clinician control device.

12. The method of claim 9, wherein the patient testing device comprises at least one of a joystick and a temperature-controlled touchpad, and wherein said displaying instructions for performing the at least one of the motor function test and the sensory test comprises:
displaying instructions to perform at least one of manipulation of the at least one joystick and touching of the temperature-controlled touchpad.

13. The method of claim 9, wherein the clinician control device comprises at least one joystick, and wherein said clinician control device is operable to provide haptic feedback via the at least one joystick corresponding to input provided via the joystick at the patient testing device.

14. The method of claim 9, wherein said displaying instructions for performing the at least one of a motor function test and a sensory test comprises displaying instructions at a patient computing device distinct from the patient testing device.

15. The method of claim 9, wherein said displaying instructions for performing the at least one of a motor function test and a sensory test comprises displaying instructions at a patient computing device for using the patient testing device, and displaying instructions at a clinician computing device for using a clinician control device, said displaying of instructions at the patient computing device and the clinician computing device being coordinated in concert in support of performance of the at least one of the motor function test and the sensory test.

16. The method of claim 9, wherein said receiving instruction data for performance of the at least one of the motor function test and the sensory test comprises receiving first instruction data at a first time for a first test and receiving second instruction data at a second time for a second test, and wherein the receiving of second instruction data at the second time for the second test is a result of sending of the second instruction data at the second time as determined automatedly according to data gathered during the first test.

17. A computer program product for implementing a method of controlling a display of a computerized device to perform remote motor function and sensory function testing, the computer program product comprising a non-transitory computer-readable medium storing executable instructions that, when executed by a processor, cause a computerized system to perform a method comprising:
receiving instruction data for performance of at least one of a motor function test and a sensory test;
displaying instructions for performing the at least one of a motor function test and a sensory test, the instructions comprising instructions to interact with a patient testing device by physical contact with the patient testing device, the patient testing device comprising at least one sensor for gathering sensor data as a result of the physical contact;
gathering sensor data corresponding to the physical contact with the patient testing device;
processing the sensor data to generate feedback data, the feedback data indicating the patient's interaction with the patient testing device and being operable to control a clinician control device to provide feedback corresponding to the patient's interaction; and
transmitting the feedback data via a communications network.

18. The computer program product of claim 17, further comprising instructions to perform the method comprising:
storing the feedback data in a memory;
wherein said transmitting the feedback data via the communications network involves retrieving the feedback data from the memory, and wherein said transmitting is performed in response to a request for transmission of the feedback data at a time after completion of the at least one of the motor function test and the sensory test.

19. The computer program product of claim 17, wherein said displaying instructions for performing the at least one of a motor function test and a sensory test comprises displaying instructions at a patient computing device for using the patient testing device, and displaying instructions at a clinician computing device for using a clinician control device, said displaying of instructions at the patient computing device and the clinician computing device being coordinated in concert in support of performance of the at least one of the motor function test and the sensory test.

20. The method of claim 17, wherein said receiving instruction data for performance of the at least one of the motor function test and the sensory test comprises receiving first instruction data at a first time for a first test and receiving second instruction data at a second time for a second test, and wherein the receiving of second instruction data at the second time for the second test is a result of sending of the second instruction data at the second time as determined automatedly according to data gathered during the first test.
